(19) United States
(12) Patent Application Publication
(10) Pub. No.: US 2014/0299857 A1
(43) Pub. Date: Oct. 9, 2014

(54) COMPLEX COMPOUND, DRYING AGENT, SEALING STRUCTURE AND ORGANIC EL ELEMENT

(71) Applicant: Futaba Corporation, Mobara-shi (JP)

(72) Inventors: Yusuke HOSHINA, Mobara-shi (JP); Satoshi TANAKA, Mobara-shi (JP)

(73) Assignee: Futaba Corporation, Mobara-shi (JP)

(21) Appl. No.: 14/246,585

(22) Filed: Apr. 7, 2014

(30) Foreign Application Priority Data
Apr. 9, 2013 (JP) 2013-081186

(51) Int. Cl. H01L 51/52; C07C 43/11; C07C 31/20; C07F 5/06; C07C 43/13

(52) U.S. Cl. CPC H01L 51/5246; C07F 5/069; C07C 43/13; C07C 31/20; C07C 43/11; H01L 51/5259
USPC 257/40; 556/182; 428/448; 428/704

(57) ABSTRACT

A complex compound obtained by reacting a compound represented by the following formula (1) and a polyol having an ether bond in a molecule and having 4 to 12 carbon atoms or a branch polyol having 5 to 7 carbon atoms:

M(OR)ₙ (1)

wherein R respectively represents an alkyl group having 4 to 12 carbon atoms or an acyl group having 2 to 12 carbon atoms, M represents an aluminum atom, a titanium atom or a silicon atom, and n represents 3 or 4.

### CROSS-REFERENCE TO RELATED APPLICATION

This application claims priority to Japanese Patent Application 2013-081186 filed on Apr. 9, 2013, all of which are herein incorporated by reference in their entirety.

### BACKGROUND OF THE INVENTION

1. Field of the Invention

The present invention relates to a complex compound, and a drying agent, a sealing structure and an organic EL element using the same.

2. Related Background Art

In recent years, research and development have been actively carried out regarding an organic EL display or an organic EL light that is a light-emitting device using an organic electroluminescence (EL) element. The organic EL element has a structure in which an organic layer that is a thin film containing an organic light-emitting material is interposed between a pair of electrodes. The organic EL element is a light-emitting element that generates excitons using holes and electrons injected into and recombined in the thin film and emits light (fluorescence or phosphorescence) generated when the excitons are deactivated.

The biggest problem with the organic EL element is improving the durability, and particularly, preventing the generation and growth of non-light-emitting sections in the organic layer which are called dark spots. When a dark spot grows to a diameter of several tens of micrometers, a non-light-emitting section becomes visually observable. As a principal cause of the dark spot, it is known that moisture and oxygen have a large influence, and particularly, moisture has a large influence even in an extremely small amount.

Therefore, a variety of studies are underway regarding a method for preventing moisture from intruding into the organic EL element, and, at the moment, it is normal to employ a hollow sealing structure in which an organic layer and electrodes are sealed in an airtight container having a dry inert gas atmosphere and, additionally, a drying agent is injected into the airtight container (for example, refer to JP 2002-33187 A).

Meanwhile, for the purpose of the physical protection of the organic layer, improvement of the heat dissipation properties, and the like, a filled sealing structure in which an airtight container of an organic EL element is filled with a filler has been proposed, and additionally, a filled sealing structure in which a drying agent is contained as a filler also has been proposed. For example, JP 2002-33187 A discloses a method in which a solution containing an organic metal compound as a drying agent is used as a filler, and JP 2012-38660 A discloses a method in which an organic metal compound having a predetermined structure which is a drying agent along with a viscous substitution material such as silicone oil is used as a filler.

### SUMMARY OF THE INVENTION

Meanwhile, in the method described in JP 2002-33187 A, it is necessary to provide a protective layer on the organic layer to avoid the influence of an organic solvent contained in the filler, and thus there is a problem of an operation becoming troublesome. On the other hand, in the method described in JP 2012-38660 A, while it is not necessary to provide a protective layer on the organic layer, since the viscous substitution material having no water-trapping properties is contained, as a whole, there is still room for improvement of water-trapping properties.

Therefore, an object of the invention is to provide a complex compound that may be used as a filler without adding an organic solvent or a viscous substitution material and may have excellent water-trapping properties, and to provide a drying agent, a sealing structure and an organic EL element using the same.

The invention provides a complex compound obtained by reacting a compound represented by the following formula (1) and a polyol having an ether bond in a molecule and having 4 to 12 carbon atoms or a branch polyol having 5 to 7 carbon atoms:

M(OR)ₙ (1)

wherein R respectively represents an alkyl group having 4 to 12 carbon atoms or an acyl group having 2 to 12 carbon atoms, M represents an aluminum atom, a titanium atom or a silicon atom, and n represents 3 or 4.

Since the above-described complex compound may have a liquid form, and may have a viscosity that can be adjusted in a range of, for example, 0.1 Pa·s to 5000 Pa·s at room temperature (25° C.), the complex compound may be used as a filler without adding an organic solvent or a viscous substitution material. Furthermore, the complex compound of the invention may have excellent water-trapping properties. In addition, the complex compound of the invention may have excellent translucency, and may not crack and become opaque even after trapping water, and therefore the complex compound may be preferably applied to a top emission-type organic EL element extracting light from a side of a sealing substrate described below.

The invention also provides a drying agent comprising the complex compound.

The invention also provides a sealing structure in which a pair of substrates are sealed with a sealing agent and the drying agent is provided in the sealing structure.

The invention also provides an organic EL element including an element substrate, a sealing substrate disposed opposite to the element substrate, a laminate being provided on the element substrate and including an organic layer interposed between a pair of electrodes, and a sealing agent sealing outer peripheral parts of the element substrate and the sealing substrate, in which a sealed space is filled with the drying agent.

According to the invention, it is possible to provide a complex compound that may be used as a filler without adding an organic solvent or a viscous substitution material and may have excellent water-trapping properties, and to provide a drying agent, a sealing structure and an organic EL element using the same.

### BRIEF DESCRIPTION OF THE DRAWINGS

FIG. 1 is a schematic cross-sectional view illustrating a configuration of an organic EL element according to an embodiment of the invention.

FIG. 2 is a schematic cross-sectional view illustrating a manufacturing process of the organic EL element according to the embodiment of the invention.

### DETAILED DESCRIPTION

Hereinafter, an embodiment of the invention will be described, but the invention is not limited thereto.

### Complex Compound

A complex compound of the present embodiment is obtained by reacting a compound represented by the following formula (1) and a polyol having an ether bond in a molecule and having 4 to 12 carbon atoms or a branch polyol having 5 to 7 carbon atoms.

M(OR)ₙ (1)

In Formula (1), R respectively represents an alkyl group having 4 to 12 carbon atoms or an acyl group having 2 to 12 carbon atoms, is preferably an alkyl group having 4 to 12 carbon atoms, more preferably an alkyl group having 4 to 8 carbon atoms, and still more preferably an alkyl group having 4 to 6 carbon atoms. The above-described alkyl group and the above-described acyl group may be linear, branched or cyclic, and a branched alkyl group is preferred, a tert-alkyl group or a sec-alkyl group is more preferred, and a sec-alkyl group is still more preferred.

Specific examples of the alkyl group having 4 to 12 carbon atoms include a butyl group, a pentyl group, a hexyl group, a heptyl group, an octyl group, a nonyl group, a decyl group, an undecyl group, a dodecyl group and the like.

Specific examples of the acyl group having 2 to 12 carbon atoms include an acetyl group, a trifluoroacetyl group, a propionyl group, a butyryl group, an isobutyryl group, a valeryl group, an isovaleryl group, a pivaloyl group, a benzoyl group and the like.

In Formula (1), M represents an aluminum atom, a titanium atom or a silicon atom, and is preferably an aluminum atom.

In Formula (1), n represents the valence of M, and is 3 in a case in which M is an aluminum atom, and is 4 in a case in which M is a titanium atom or a silicon atom.

Specific examples of the compound represented by Formula (1) include aluminum-n-butoxide, aluminum-sec-butoxide, aluminum-tert-butoxide, aluminum-n-octoxide, aluminum-sec-octoxide, aluminum-n-dodecoxide, aluminum-sec-dodecoxide, titanium-n-butoxide, titanium-sec-butoxide, titanium-tert-butoxide, titanium-n-octoxide, titanium-sec-octoxide, titanium-n-dodecoxide, titanium-sec-dodecoxide, tetra-n-butoxysilane, tetra-sec-butoxysilane, tetra-tert-butoxysilane, tetra-n-octoxysilane, tetra-sec-octoxysilane, tetra-n-dodecoxysilane, tetra-sec-dodecoxysilane, and the like.

Meanwhile, the compound represented by Formula (1) may be an association of the compounds represented by Formula (1).

The number of carbon atoms in the polyol having an ether bond in the molecule and having 4 to 12 carbon atoms is preferably in a range of 4 to 10, more preferably in a range of 4 to 8, and still more preferably in a range of 4 to 6. The number of the ether bonds in the polyol is preferably in a range of 1 to 3, more preferably one or two, and still more preferably one. The number of hydroxyl groups in the polyol is preferably in a range of 2 to 4, more preferably two or three, and still more preferably two (diol). Meanwhile, the polyol may be linear, branched or cyclic.

Specific examples of the polyol having an ether bond in the molecule and having 4 to 12 carbon atoms include diethylene glycol, dipropylene glycol, dibutylene glycol, dipentylene glycol, triethylene glycol, tetraethylene glycol, tripropylene glycol, tetrapropylene glycol, and the like, and diethylene glycol or dipropylene glycol is preferred.

The number of the hydroxyl groups in the branch polyol having 5 to 7 carbon atoms is preferably in a range of 2 to 4, more preferably two or three, and still more preferably two (diol). Specific examples of the polyol include hexylene glycol (2-methyl-2,4-pentanediol), 2,2-dimethyl-1,3-propanediol, 2,3-dimethyl-2,3-butanediol, 2-methyl-1,3-hexanediol, trimethylolpropane, pentaerythritol, and the like, and hexylene glycol is preferred.

The conditions for reacting the compound represented by Formula (1) and the polyol having an ether bond in the molecule and having 4 to 12 carbon atoms or the branch polyol having 5 to 7 carbon atoms (hereinafter, the above-described two kinds of polyols will be referred to simply as “polyol”) can be appropriately selected in accordance with a raw material being used, but the compound and the polyol are preferably reacted under, for example, solvent-free reflux conditions. Meanwhile, in a case in which the compound and the polyol are reacted in the presence of a solvent, it is possible to distill the solvent at a reduced pressure after completing the reaction.

As the compound represented by Formula (1) and the polyol, one kind of them may be used alone, or two or more kinds of them may be used in combination, and it is preferably to use one kind of them respectively.

The compound represented by Formula (1) and the polyol can be reacted at an arbitrary ratio, and it is possible to adjust the viscosity by changing the ratio. The ratio can be set to, for example, 0.1 moles to 1 mole of the polyol, preferably, 0.2 moles to 0.8 moles of the polyol based on 1 mole of the compound represented by Formula (1).

While the structure of the complex compound obtained by reacting the compound represented by Formula (1) and the polyol is not evident, the complex compound is assumed to have a structure in which some or all of the OR groups bonded to M in the compound represented by Formula (1) are substituted by a polyol-derived alkoxy group.

In addition, while the reason for the excellent water-trapping properties of the complex compound is not evident either, the present inventors assume as follows.

That is, when the complex compound comes into contact with water, the OR group or the polyol-derived alkoxy group in the complex compound is substituted by a water-derived hydroxyl group, and therefore water is incorporated into the complex compound. Since the complex compound of the invention includes a relatively large number of OR groups and alkoxy groups in the unit amount, the water-trapping properties are considered to be excellent.

### Drying Agent

A drying agent of the embodiment comprises the above-described complex compound. Meanwhile, the drying agent of the embodiment may contain a resin such as silicone or may be jointly used with other drying agents within the scope of the effects of the invention.

The drying agent of the embodiment can be applied to a subject using, for example, an application method using a dispense, an one drop fill (ODF) method, a screen printing method, a spraying method, a hot melting method or the like. In a case in which the application method using a dispense is applied, the viscosity of the drying agent is preferably in a range of 1 Pa·s to 5000 Pa·s, more preferably in a range of 1 Pa·s to 1000 Pa·s, and still more preferably in a range of 1 Pa·s to 300 Pa·s. In addition, in a case in which the ODF method is applied, the viscosity of the drying agent is preferably in a range of 0.1 Pa·s to 1 Pa·s.

According to the drying agent of the embodiment, it may be possible to set the water-trapping volume to 10 wt % or more and preferably 15 wt % or more, and the water-trapping properties may be favorable compared with those of a drying agent that can be filled of the related art.

### Sealing Structure

A sealing structure of the embodiment is a sealing structure in which a pair of substrates is sealed with a sealing agent, and the above-described drying agent is provided in the sealing structure. The drying agent may be applied only to some of a sealed space, for example, a predetermined place on a substrate, or may fill the sealed space.

The sealing structure of the embodiment can be particularly preferably used to seal a device that is easily affected by moisture. Examples of the above-described device include organic electronic devices such as an organic EL element, an organic semiconductor and an organic solar cell.

### Organic EL Element

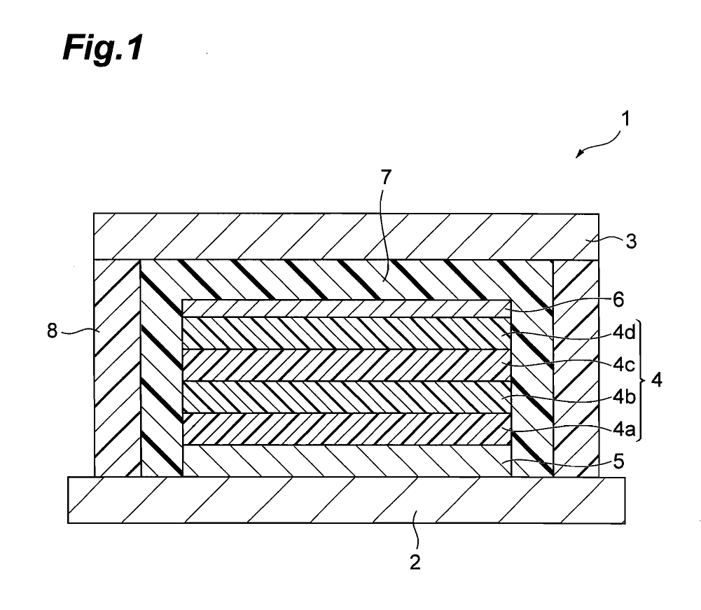

Hereinafter, an embodiment of an organic EL element of the invention will be described on the basis of FIG. 1.

An organic EL element 1 of the present embodiment is an organic EL element having a filled sealing structure constituted of an element substrate 2, a sealing substrate 3 disposed opposite to the element substrate 2, a laminate being provided on the element substrate 2 and including an organic layer 4 interposed between a pair of electrodes 5 and 6, a sealing agent 8 sealing outer peripheral parts of the element substrate 2 and the sealing substrate 3, and a filler 7 filling a sealed space. The filler 7 is the above-described drying agent of the embodiment.

As the elements of the organic EL element 1 except for the filler 7, it is possible to apply elements in a well-known organic EL element of the related art, and an example thereof will be briefly described below.

The element substrate 2 is made of a rectangular insulating and translucent glass substrate, and a positive electrode (electrode) 5 is formed on the element substrate 2 by using indium tin oxide (ITO) that is a transparent conductive material. The positive electrode 5 is formed by patterning an ITO film formed on the element substrate 2 using, for example, a physical vapor deposition (PVD) method such as a vacuum deposition method or a sputtering method through etching using a photoresist method in a predetermined pattern shape. A part of the positive electrode 5 as the electrode is connected to a drive circuit (not illustrated) extracted up to an end section of the element substrate 2.

On a top surface of the positive electrode 5, the organic layer 4 that is a thin film containing an organic light-emitting material is laminated using, for example, a PVD method such as a vacuum deposition method or a resistance heating method. The organic layer 4 may be formed of a single layer or may be formed of a plurality of layers having different functions. The organic layer 4 in the embodiment is a four-layer structure in which a hole-injecting layer 4a, a hole-transporting layer 4b, a light-emitting layer 4c and an electron-transporting layer 4d are sequentially laminated from the positive electrode 5 side. The hole-injecting layer 4a is formed of, for example, a copper phthalocyanine (CuPc) thin film having a thickness of several tens of nanometers. The hole-transporting layer 4b is formed of, for example, a bis[N-(1-naphthyl)-N-phenyl]benzidine(a-NPD) thin film having a thickness of several tens of nanometers. The light-emitting layer 4c is formed of, for example, a tris(8-quinolinolato)aluminum (Alq3) thin film having a thickness of several tens of nanometers. The electron-transporting layer 4d is formed of, for example, a lithium fluoride (LiF) thin film having a thickness of several nanometers. In addition, a light-emitting section is formed using a laminate in which the positive electrode 5, the organic layer 4 and a negative electrode 6 described below are sequentially laminated.

On a top surface of the organic layer 4 (electron-transporting layer 4d), the negative electrode (electrode) 6 that is a metal thin film is laminated using a PVD method such as a vacuum deposition method. Examples of a material for the metal thin film include a metal having a small work function such as Al, Li, Mg or In, an alloy having a small work function such as Al—Li or Mg—Al, and the like. The negative electrode 6 is formed of, for example, a thin film having a thickness in a range of several tens of nanometers to several hundreds of nanometers (preferably in a range of 50 nm to 200 nm). A part of the negative electrode 6 is connected to the drive circuit (not illustrated) extracted up to an end section of the element substrate 2.

The sealing substrate 3 is disposed opposite to the element substrate 2 with the organic layer 4 therebetween, and the outer peripheral parts of the element substrate 2 and the sealing substrate 3 are sealed with the sealing agent 8. As the sealing agent, for example, an ultraviolet curable resin can be used. Furthermore, a seal space is filled with the filler 7 that is the drying agent of the embodiment. Therefore, the organic layer 4 and the like are protected.

Meanwhile, the above-described organic EL element is a bottom emission-type organic EL element that extracts light from the element substrate side, but the organic EL element of the invention may be a top emission-type organic EL element that extracts light from the sealing substrate side. While the top emission-type organic EL element can be also manufactured using a well-known method of the related art, it is necessary to make some changes such as use of a substrate having translucency for the sealing substrate 3 and use of a transparent electrode for the negative electrode 6 or exchange of the locations of the positive electrode 5 and the negative electrode 6. The drying agent of the embodiment has excellent translucency, and does not crack and become opaque even after trapping water, and therefore the drying agent can be particularly preferably used for the top emission-type organic EL element.

### Manufacturing Method for Organic EL Element

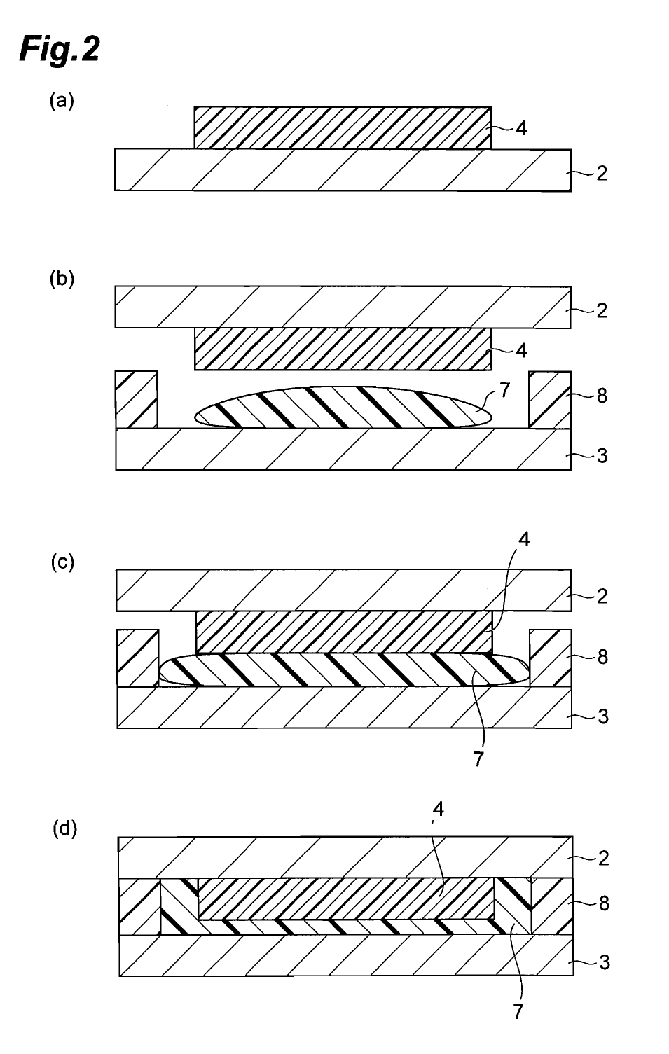

Hereinafter, a manufacturing process, particularly, sealing process for the above-described organic EL element will be described on the basis of FIG. 2.

First, a laminate obtained by laminating the organic layer 4 and the like (electrodes are not illustrated in the drawing) on the element substrate 2 is provided (FIG. 2(a)).

Next, a sufficient volume of the drying agent of the embodiment that can fill a sealed space is applied onto the separately-provided sealing substrate 3 using a dispenser. Furthermore, the sealing agent 8 is applied using a dispenser so as to surround the drying agent applied on the sealing substrate 3 (FIG. 2(b)). The above-described operations are preferably carried out in a glove box substituted by nitrogen having a dew point of −76° C. or lower.

Next, the element substrate 2 on which the organic layer 4 and the like are laminated and the sealing substrate 3 are adhered to each other (FIG. 2(c)). The adhered substrates are sealed through UV radiation and heating at approximately 80° C., thereby manufacturing the organic EL element of the embodiment (FIG. 2(d)).

### EXAMPLES

Hereinafter, the invention will be specifically described using examples. However, the invention is not limited to the examples.

Meanwhile, in the embodiment, the viscosity and the water-trapping volume were measured using the following methods.

### Viscosity

The viscosity at 25° C. was measured using an HBDV-E digital viscometer manufactured by Brookfield Engineering.

### Water-Trapping Volume

A sample was added to a water-containing ethanol having a water content ratio of 5 mass % so that the sample concentration reached 10 mass %. The solution was stirred for one minute, and then, additionally, centrifugally separated under conditions of at 2000 rpm and for 10 minutes. A change in the water content ratio of the ethanol after the centrifugal separation was computed as the water-trapping amount using a KF method moisture meter (CA-100, VA-100: vaporization method), and the water-trapping volume was computed on the basis of the following formula.

Water-trapping volume [wt %] =water-trapping amount [mg]/sample amount [mg]

### Preparation of the Complex Compound

### Example 1

Aluminum-sec-butoxide and dipropylene glycol were injected into an eggplant flask at a molar ratio of 1:0.5, and heated and refluxed at 130° C. for one hour, thereby obtained a liquid-form complex compound. The obtained complex compound had a viscosity of 235 Pa·s and a water-trapping volume of 18.7 wt %.

### Example 2

Aluminum-sec-butoxide and dipropylene glycol were injected into an eggplant flask at a molar ratio of 1:0.25, and heated and refluxed at 130° C. for one hour, thereby obtained a liquid-form complex compound. The obtained complex compound had a viscosity of 7.2 Pa·s and a water-trapping volume of 18.1 wt %.

### Example 3

Aluminum-sec-butoxide and hexylene glycol were injected into an eggplant flask at a molar ratio of 1:0.5, and heated and refluxed at 130° C. for one hour, thereby obtained a liquid-form complex compound. The obtained complex compound had a viscosity of 0.94 Pa·s and a water-trapping volume of 19.4 wt %.

### Example 4

Aluminum-sec-butoxide and hexylene glycol were injected into an eggplant flask at a molar ratio of 1:0.75, and heated and refluxed at 130° C. for one hour, thereby obtained a liquid-form complex compound. The obtained complex compound had a viscosity of 1.8 Pa·s and a water-trapping volume of 19.9 wt %.

### Example 5

Aluminum-sec-butoxide and diethylene glycol were injected into an eggplant flask at a molar ratio of 1:0.25, and heated and refluxed at 130° C. for one hour, thereby obtained a liquid-form complex compound. The obtained complex compound had a viscosity of 8.99 Pa·s and a water-trapping volume of 18.9 wt %.

### Comparative Examples 1 to 5

Complex compounds were synthesized in the same manner as in Example 1 except for the fact that ethylene glycol (Comparative Example 1), propylene glycol (Comparative Example 2), 1,4-butanediol (Comparative Example 3), 1,5-pentanediol (Comparative Example 4) and 1,3-octylene glycol (Comparative Example 5) were used instead of dipropylene glycol. In all the comparative examples, solid was precipitated, and it was not possible to obtain a liquid-form complex compound.

What is claimed is:

1. A complex compound obtained by reacting a compound represented by the following formula (1) and a polyol having an ether bond in a molecule and having 4 to 12 carbon atoms or a branch polyol having 5 to 7 carbon atoms:
M(OR)ₙ (1)
wherein R respectively represents an alkyl group having 4 to 12 carbon atoms or an acyl group having 2 to 12 carbon atoms, M represents an aluminum atom, a titanium atom or a silicon atom, and n represents 3 or 4.

2. The complex compound according to claim 1, wherein R is an alkyl group having 4 to 12 carbon atoms.

3. The complex compound according to claim 1, wherein R is an alkyl group having 4 to 8 carbon atoms.

4. The complex compound according to claim 1, wherein R is an alkyl group having 4 to 6 carbon atoms.

5. The complex compound according to claim 1, wherein M is an aluminum atom.

6. The complex compound according to claim 1, wherein the number of carbon atoms in the polyol having an ether bond in the molecule and having 4 to 12 carbon atoms is in a range of 4 to 10.

7. The complex compound according to claim 1, wherein the number of carbon atoms in the polyol having an ether bond in the molecule and having 4 to 12 carbon atoms is in a range of 4 to 8.

8. The complex compound according to claim 1, wherein the number of carbon atoms in the polyol having an ether bond in the molecule and having 4 to 12 carbon atoms is in a range of 4 to 6.

9. The complex compound according to claim 1, wherein the number of the ether bonds in the polyol having an ether bond in the molecule and having 4 to 12 carbon atoms is in a range of 1 to 3.

10. The complex compound according to claim 1, wherein the number of the ether bonds in the polyol having an ether bond in the molecule and having 4 to 12 carbon atoms is one or two.

11. The complex compound according to claim 1, wherein the number of the ether bonds in the polyol having an ether bond in the molecule and having 4 to 12 carbon atoms is one.

12. The complex compound according to claim 1, wherein the number of hydroxyl groups in the polyol having an ether bond in the molecule and having 4 to 12 carbon atoms is in a range of 2 to 4.

13. The complex compound according to claim 1, wherein the number of hydroxyl groups in the polyol having an ether bond in the molecule and having 4 to 12 carbon atoms is two or three.

14. The complex compound according to claim 1, wherein the number of hydroxyl groups in the polyol having an ether bond in the molecule and having 4 to 12 carbon atoms is two.

15. The complex compound obtained by reacting a compound represented by the following formula (1) and a polyol having an ether bond in a molecule and having 4 to 8 carbon atoms or a branch polyol having 5 to 7 carbon atoms:
M(OR)ₙ (1)
wherein R respectively represents an alkyl group having 4 to 10 carbon atoms, M represents an aluminum atom, a titanium atom or a silicon atom, and n represents 3 or 4.

16. The complex compound obtained by reacting a compound represented by the following formula (1) and a polyol having an ether bond in a molecule and having 4 to 6 carbon atoms or a branch polyol having 5 to 7 carbon atoms:
M(OR)ₙ (1)
wherein R respectively represents an alkyl group having 4 to 6 carbon atoms, M represents an aluminum atom, and n represents 3.

17. A drying agent comprising the complex compound according to claim 1.

18. A sealing structure,
wherein a pair of substrates are sealed with a sealing agent, and the drying agent according to claim 17 is provided in the sealing structure.

19. An organic EL element comprising:
an element substrate;
a sealing substrate disposed opposite to the element substrate;
a laminate being provided on the element substrate and including an organic layer interposed between a pair of electrodes; and
a sealing agent sealing outer peripheral parts of the element substrate and the sealing substrate,
wherein a sealed space is filled with the drying agent according to claim 17.
